(19) United States
(12) Patent Application Publication
(10) Pub. No.: US 2021/0219632 A1
(43) Pub. Date: Jul. 22, 2021

(54) HEATING AND COOLING GARMENT SYSTEM

(71) Applicant: Cipher Skin, Denver, CO (US)

(72) Inventor: Phillip Bogdanovich, Evergreen, CO (US)

(73) Assignee: Cipher Skin, Denver, CO (US)

(21) Appl. No.: 17/143,843

(22) Filed: Jan. 7, 2021

Related U.S. Application Data
(60) Provisional application No. 62/958,042, filed on Jan. 7, 2020.

(51) Int. Cl. A41D 1/00; A41D 13/005; F25B 21/04

(52) U.S. Cl. CPC A41D 1/002; A41D 13/0051; F25B 2600/07; F25B 21/04; A41D 2400/12; A41D 13/0053

(57) ABSTRACT

In one example, a heating and cooling garment system includes a garment, and a plurality of heating and cooling zones positioned within a portion of the garment. Each heating and cooling zone is configured to independently heat or cool based on heating and/or cooling instructions received from a mobile application executed on a wireless device.

### CROSS-REFERENCE TO RELATED APPLICATION

This application claims priority to U.S. Provisional Patent Application No. 62/958,042 filed Jan. 7, 2020, the entirety of which is incorporated herein by reference.

### TECHNICAL FIELD

This invention relates generally to heating and cooling systems, and more particularly to a heating and cooling garment system

### BACKGROUND

Traditionally, a user has utilized a heating pack (or device) to heat a portion of their body (e.g., apply heat to muscles in the user's back), and then the user has used a different cooling pack (or device) to cool the same portion of their body (e.g., apply cold to muscles in the user's back). These traditional systems, however, may be deficient.

### SUMMARY

In one example, a heating and cooling garment system includes a garment, and a plurality of heating and cooling zones positioned within a portion of the garment. Each heating and cooling zone is configured to independently heat or cool based on heating and/or cooling instructions received from a mobile application executed on a wireless device.

In another example, a method includes receiving heating and/or cooling instructions from a mobile application executed on a wireless device. The method further includes independently heating or cooling each of a plurality of heating and cooling zones positioned within a portion of a garment, based on the heating and/or cooling instructions.

In a further example, a heating and cooling sleeve includes a sleeve configured to be in contact with a user's skin, a plurality of a plurality of heating and cooling zones positioned in series within the sleeve, and one or more processors positioned within the sleeve. Each heating and cooling zone includes a Peltier heating and cooling device, and a propagation strip thermally coupled to a first side of the Peltier heating and cooling device. The processors are operable, upon execution, to receive heating and/or cooling instructions from a wireless device. The heating and/or cooling instructions include an instruction to heat or cool a first heating and cooling zone of the plurality of heating and cooling zones to a first temperature and further include an instruction to heat or cool a second heating and cooling zone of the plurality of heating and cooling zones to a second different temperature. The first heating and cooling zone is configured to independently heat or cool to the first temperature based on the heating and/or cooling instructions received from the wireless device. The second heating and cooling zone is configured to independently heat or cool to the second different temperature based on the heating and/or cooling instructions received from the wireless device.

### BRIEF DESCRIPTION OF THE FIGURES

For a more complete understanding of the present disclosure and its features and advantages, reference is now made to the following description, taken in conjunction with the accompanying drawings, in which:

FIG. 1 illustrates one example of a heating and cooling garment system.

FIG. 2 illustrates another example of a heating and cooling garment system.

FIG. 3 illustrates one example of one or more layers of the heating and cooling garment system.

FIG. 4 illustrates one example of heating and cooling zones included on a heating/cooling layer of the heating and cooling garment system.

FIG. 5 illustrates one example of a mesh-patterned monitoring system of the heating and cooling garment system.

FIG. 6 illustrates one example of a power layer of the heating and cooling garment system.

FIG. 7 illustrates one example of an insulation layer of the heating and cooling garment system.

FIG. 8 illustrates an alternative example of the monitoring system of the heating and cooling garment system.

### DETAILED DESCRIPTION

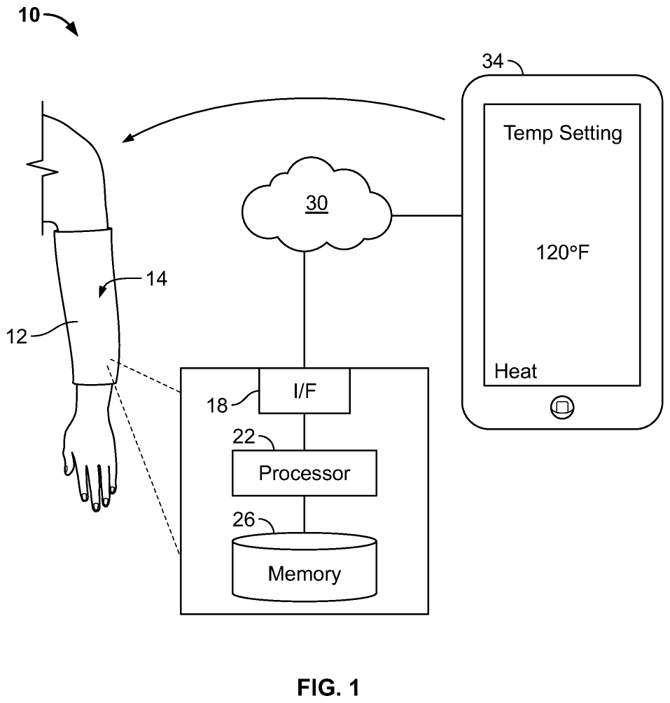

Embodiments of the present disclosure are best understood by referring to FIGS. 1-8 of the drawings, like numerals being used for like and corresponding parts of the various drawings.

FIG. 1 illustrates one example of a heating and cooling garment system 10. In the illustrated example, the system 10 includes a garment 12. The garment 12 represents any item that may be worn, carried, or used to adorn a user. For example, the garment 12 may be a shirt, pants, a towel, a sleeve, leggings, compression gear (e.g., compression sleeve, compression leggings), any other item that may be worn, carried, or used to adorn a user, or any combination of the preceding. In other examples, the garment 12 may be a mat (e.g., a yoga mat), a stretching device (e.g., a foam roll), any other item that may be used by a user, or any combination of the preceding. In the illustrated example, the garment 12 is a sleeve that may be worn (or otherwise applied) on a user's arm.

In the example illustrated in FIG. 1, the garment 12 includes a heating and cooling system 14. The heating and cooling system 14 represents any system that may heat and/or cool a portion of the garment 12. In some examples, the heating and cooling system 14 is a thermoelectric heating and cooling system that can heat and/or cool a portion of the garment 12 in response to electrical current. As one example of this, the thermoelectric heating and cooling system is a Peltier thermoelectric heating and cooling system that utilizes the Peltier effect to heat and/or cool in response to electrical current. The heating and cooling system 14 may heat and/or cool any portion of the garment 12. For example, it may heat and/or cool all of the garment 12 or only a portion of the garment 12. The heating and cooling system 14 may heat and/or cool a portion of the garment 12 to any temperature. For example, the heating and cooling system 14 may heat a portion of the garment 12 to any temperature above room temperature (i.e., 68-72° F.), such as a temperature within a range of 72° F. and 125° F. As another example, the heating and cooling system 14 may cool a portion of the garment 12 to any temperature below room temperature (i.e., 68-72° F.), such as a temperature within a range of 30° F. and 68° F. In some examples, the heating and cooling system 14 may heat and/or cool a portion of the garment 12 to any temperature within a range of 36° F. and 392° F.

In the illustrated example, the heating and cooling system 14 (and/or the garment 12) includes a network interface 18, a processor 22, and a memory unit 26. Network interface 18 is operable to receive information from a network 30, transmit information through network 30, perform processing of information, communicate to other devices, or any combination of the preceding. Network interface 18 represents any port or connection, real or virtual, including any suitable hardware and/or software, including protocol conversion and data processing capabilities, to communicate through a local area network (LAN), a metropolitan area network (MAN), a wide area network (WAN), or other communication system that allows garment 12 to exchange information with network 30, wireless device 34, or other components of system 10.

Processor 22 communicatively couples to network interface 18 and memory unit 26, and controls the operation and administration of heating and cooling system 14 by processing information received from network interface 18 and memory unit 26. Processor 22 includes any hardware and/or software that operates to control and process information. For example, processor 22 may execute instructions in order to cause heating and cooling system 14 to heat and/or cool a portion of the garment 12. As another example, processor 22 may execute instructions in order to communicate with a wireless device 34, so as to receive heating/cooling instructions from the wireless device 34 and/or to provide data (e.g., biometric data) to the wireless device 34. Processor 22 may be a programmable logic device, a microcontroller, a microprocessor, any suitable processing device, or any combination of the preceding.

Memory unit 26 stores, either permanently or temporarily, data, operational software, or other information for processor 22. Memory unit 26 includes any one or a combination of volatile or non-volatile local or remote devices suitable for storing information. For example, memory unit 26 may include random access memory (RAM), read only memory (ROM), magnetic storage devices, optical storage devices, any other suitable information storage device, or any combination of the preceding. Memory unit 26 may include any suitable information for use in the operation of garment 12 and the heating and cooling system 14. Furthermore, memory unit 26 may include heating/cooling instructions received from the wireless device 34 (e.g., a schedule for heating and cooling), and may further include biometric data sensed by the garment 12 (as is discussed below).

In the illustrated example, the heating and cooling garment system 10 also includes the network 30. Network 30 represents any suitable network operable to facilitate communication between the components of system 10, such as garment 12 and wireless device 34. Network 30 may include any interconnecting system capable of transmitting audio, video, signals, data, messages, or any combination of the preceding. Network 30 may include all or a portion of a public switched telephone network (PSTN), a public or private data network, a LAN, a MAN, a WAN, a WPAN, a local, regional, or global communication or computer network, such as the Internet, a wireline or wireless network, an enterprise intranet, or any other suitable communication link, including combinations thereof, operable to facilitate communication between the components, but not limited to include data transfer to remote services (e.g., cloud data storage). Preferable examples of network 30 may include a WPAN (which may include, for example, BLUETOOTH, BLUETOOTH low power, BLUETOOTH 5, ANT+, ZIGBEE (IEEE 802.15.4), other IEEE 802.15 protocols, IEEE 802.11 A, B or G without limitation, and WI-FI (IEEE 802.11)), a cellular communication network, an infrared communication network, any other wireless network operable to facilitate communication between the components, or any combination of the preceding.

In the example illustrated in FIG. 1, the heating and cooling garment system 10 further includes the wireless device 34. Wireless device 34 represents any suitable components that may communicate with the processor 22 (and/or the garment 12 and/or the heating and cooling system 14) so as to cause the heating and cooling system 14 to heat and/or cool a portion of the garment 12. Additionally, the wireless device 34 may further communicate with a user (via a user interface) so as to allow a user to select whether to heat and/or cool the garment 12. Wireless device 34 may be a laptop, a mobile telephone or cellular telephone (such as a Smartphone), an electronic notebook, a tablet (such as an IPAD), a personal digital assistant, a remote control, any other device capable of receiving, processing, storing, and/or communicating information with other components of system 10, or any combination of the preceding.

The wireless device 34 may allow the user to display and control the heating and/or cooling of the garment 12. For example, the user may utilize a mobile device application (e.g., a mobile “app”), a web application, or other software on the wireless device 34 in order to communicate with the garment 12. In doing so, the user may send heating/cooling instructions to the garment 12, which may control the heating and/or cooling of the garment 12. For example, the user may send an instruction to heat the garment 12 to a particular temperature (e.g., 120° F.). In response to the instruction, the heating and cooling system 14 may heat all or a portion of the garment 12 to 120° F. As another example, the user may send an instruction to cool the garment 12 to a particular temperature (e.g., 32° F.). In response to the instruction, the heating and cooling system 14 may cool all or a portion of the garment 12 to 32° F.

The heating and cooling garment system 10 allows the user to switch (e.g., rapidly switch) between applying heat and applying cold to a person's body via the garment 12, without requiring the person to remove the garment 12, in some examples. Contrary to this, traditional systems may have required the person to switch systems in order to heat and then cool (e.g., use a heat pack first, then switch to a cold pack). Furthermore, the heating and cooling garment system 10 also allows the user to apply heat and apply cold to a person's body at a particular temperature (e.g., 120° F., 32° F., etc.), in some examples. Thus, it may allow for specific temperature control of a wearable device. Furthermore, it may allow a user to increase and/or decrease the output temperature of the heating and cooling garment system 10, so as to apply hot or cold to a user's body (e.g., to a user's limb).

This may allow a physical therapist to prescribe heat and cold treatment for an elbow, and the heating and cooling garment system 10 may provide this modality from the same garment 12 without the garment 12 being removed or components being changed, in some examples. The heating and cooling garment system 10 can also be worn for long periods of time without losing its effectiveness, in some examples, because the heating and cooling system 14 may utilize an electrical effect (as opposed to mechanical freezing or heating). In some examples, the heating and cooling garment system 10 may also allow for the monitoring of vital signs relative to the thermal changes against the body (e.g., biometric data), as is discussed further below. Those metrics can be transmitted to the wireless device 34 for a full view of device internal temp against the skin, skin temp, operating time, and the physiological changes associated with the modality.

The heating and cooling garment system 10 is, in some examples, a portable heating/cooling system. For example, the components used to heat and/or cool the garment 12 (e.g., the heating and cooling system 14) and the components used to communicate with the wireless device 34 (e.g., the processor 22) may be self-contained within (i.e., on or in) the garment 12. This may allow the heating and cooling garment system 10 to be lightweight, fully mobile, and tubeless (e.g., it does not require water or a circulation pump, as in traditional cooling systems). Thus, a user can wear (and utilize) the heating and cooling garment system 10 at any time, such as when the user is walking around, exercising, doing any other activity, or any combination of the preceding. In some examples, this may provide advantages over traditional systems, which may require bulky, standalone cooling units attached to the body via flexible tubes which circulate water to and from the cooling device. This traditional system, due to the inconvenience and bulk of the external tubing and detached pump/temp control unit, is not mobile. Instead, it requires a user to sit in one place and avoid snagging or tripping over the tubing while the traditional system is being used.

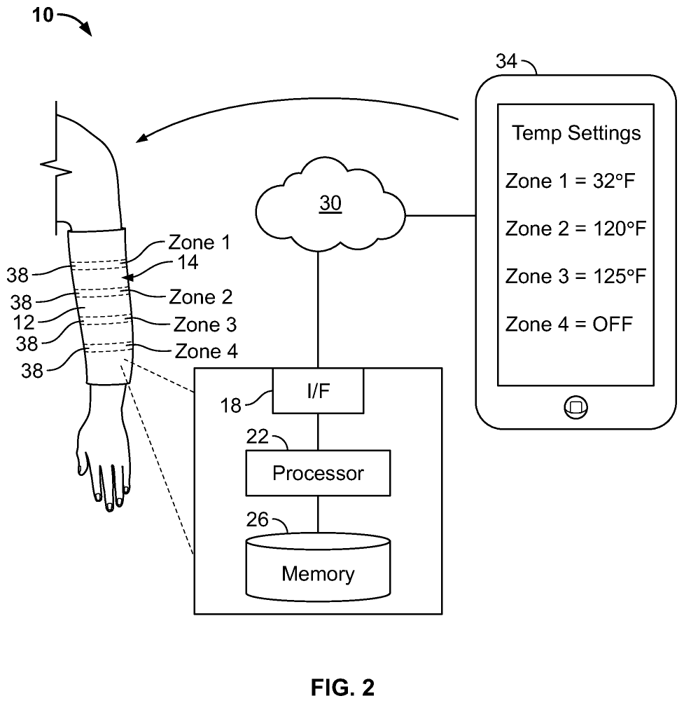

FIG. 2 illustrates another example of the heating and cooling garment system 10. The heating and cooling garment system 10 of FIG. 2 may be substantially similar to that of FIG. 1. However, the heating and cooling garment system 10 of FIG. 2 further includes heating and cooling zones 38, in some examples. The heating and cooling zones 38 may allow particular portions (or zones) of the garment 12 to be heated and/or cooled independently of other zones. For example, Zone 1 of FIG. 2 may be independently cooled to 32° F.; Zone 2 of FIG. 2 may be independently heated to 120° F., Zone 3 of FIG. 2 may be independently heated to 125° F., and Zone 4 of FIG. 2 may be turned off (i.e., it may not be heated or cooled at all). This may allow a user to use the heating and cooling garment system 10 to apply heat and/or cold to a particular portion of a user's body, without requiring the entire garment 12 to be heated and/or cooled to that same temperature. As such, a user can heat and/or cool their elbow, without doing the same to any other portion of their arm. This may allow heating and cooling treatments to be selected for individual parts of a person's body (e.g., the elbow can be heated, while the wrist is cooled, while the bicep is neither cooled nor heated).

The heating and cooling garment system 10 may include any number of heating and cooling zones 38. Also, the heating and cooling zones 38 may have any dimensions. For example, a heating and cooling zone 38 may entirely encircle a portion of the garment 12, as is illustrated in FIG. 2. Furthermore, the heating and cooling zones 38 may be positioned within (i.e., on or in) the garment 12 in any manner and/or configuration. For example, as is illustrated in FIG. 2, the heating and cooling zones 38 may be positioned in series with each other. Each heating and cooling zone 38 may be formed in any manner. For example, as is discussed below, each heating and cooling zone 38 may be formed by a Peltier device (or other thermoelectric device) thermally coupled to a propagation strip. In other examples, each heating and cooling zone 38 may be formed by a Peltier device (or other thermoelectric device) without a propagation strip. Such an example may create smaller heating and cooling zones 38, but may allow for even more targeted heating and/or cooling, in some examples.

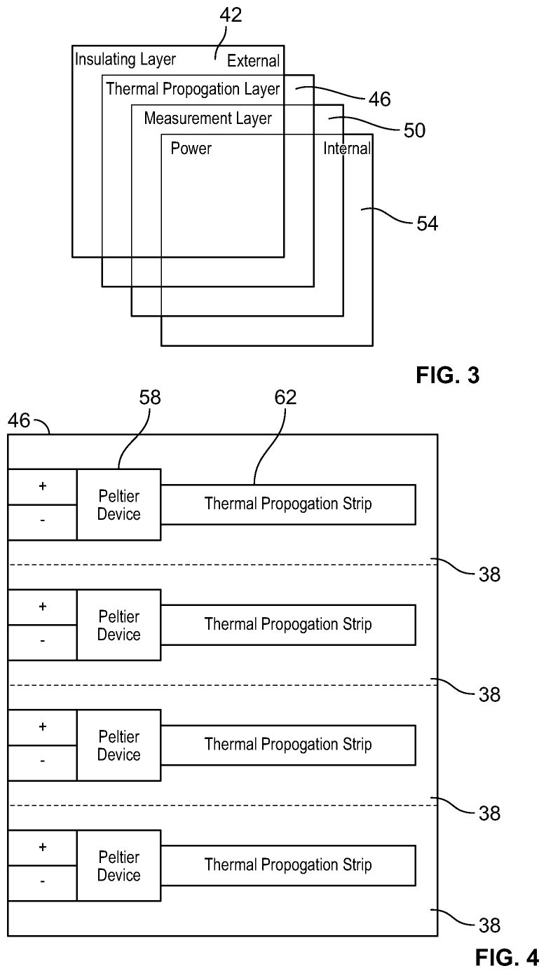

FIG. 3 illustrates one example of one or more layers of the heating and cooling garment system 10. In the example illustrated in FIG. 3, the heating and cooling garment system 10 includes four layers: an insulation layer 42, a heating/cooling layer 46, a monitoring layer 50, and a power layer 54. Although FIG. 3 illustrates the heating and cooling garment system 10 as including four layers, the heating and cooling garment system 10 may include any number of layers. For example, it may have more layers. As another example, it may have less layers. As one example of this, the heating and cooling garment system 10 may not include the monitoring layer 50.

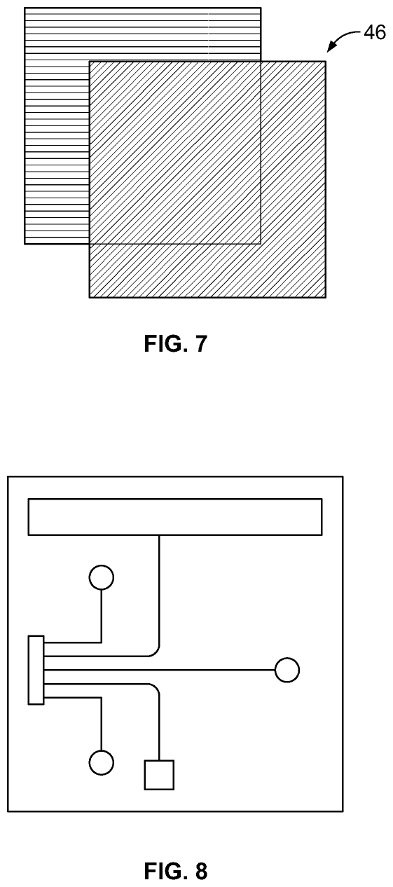

The insulation layer 46 may be the outermost layer of the heating and cooling garment system 10. As such, it may face outward (e.g., away from the user's skin) when the heating and cooling garment system 10 is being worn by the user. The insulation layer 46 may protect the components of the heating and cooling garment system 10. The insulation layer 46 may be pliable. One example of the insulation layer 46 is discussed below with regard to FIG. 7.

The heating/cooling layer 46 may provide the heating and/or cooling in the heating and cooling garment system 10. As is illustrated in FIG. 4, the heating/cooling layer 46 may include heating and cooling zones 38. Each of these heating and cooling zones 38 may be formed by a Peltier device (or other thermoelectric device) thermally coupled to a propagation strip. In other examples, each heating and cooling zone 38 may be formed by a Peltier device (or other thermoelectric device) without a propagation strip. In the example illustrated in FIG. 4, each heating and cooling zone 38 is formed by a Peltier device (or other thermoelectric device) 58 thermally coupled to a propagation strip 62.

The Peltier device 58 represents a device that may utilize electrical current to generate heat and/or cold based on the Peltier effect. Examples of the Peltier device 58 may include a Peltier heat pump, solid state refrigerator, thermoelectric cooler (TEC), or a Peltier device. In some examples, the Peltier device 58 may transfer heat from one side of the device to the other, with consumption of electrical energy, depending on the direction of the current. In such an example, applying current in one direction may cause a first side of the Peltier device 58 to generate heat (while the second side generates cold). Applying current in the opposite direction may cause the first side of the Peltier device 58 to generate cold (while the second side generates heat).

Propagation strip 62 represents a thermally conductive strip (or other thermal coupler) that may receive heat and/or cold from the first side of the Peltier device 58, and that may conduct that heat and/or cold along the dimensions of the propagation strip 62. The propagation strip 62 may be thermally coupled to the first side of the Peltier device 58, allowing it to receive the heat and/or cold generated by the first side of the Peltier device 58. The propagation strip 62 may have any size and/or shape. This size and/or shape may define the size and/or shape of the heating and cooling zone 38. The propagation strip 62 may be made of any thermally conductive material. For example, the propagation strip 62 may be made of a thin flexible metal.

The monitoring layer 50 (as shown in FIG. 3) may include a monitoring system that is capable of acquiring, compiling, analyzing, and transmitting biometric data in near real time/real time. The monitoring system may be capable of monitoring multiple biometric responses including, but not limited to: skin temperature, heating and cooling system 10 temperature, core temperature, respirations, heart rate, swelling, motion, oxygen saturation, predicted tidal volume, chest wall movement, abdominal movement in conjunction with inspiration, abdominal movement in conjunction with expiration, HRR (heart rate reserve), HRV (heart rate variability), body position relevant to perpendicular, shoulder position relevant to hip position, general body posture, up time, down time, and malfunctions. The biometric data may be transmitted to the wireless device 34 for display to the user. Examples of this monitoring system are included in U.S. Pat. Nos. 9,918,674; 9,793,952; 9,566,033; and U.S. patent application Ser. No. No. 16/834,821, entitled “Super Modular Monitoring System” and filed on Mar. 30, 2020, all of which are incorporated in their entirety herein by reference.

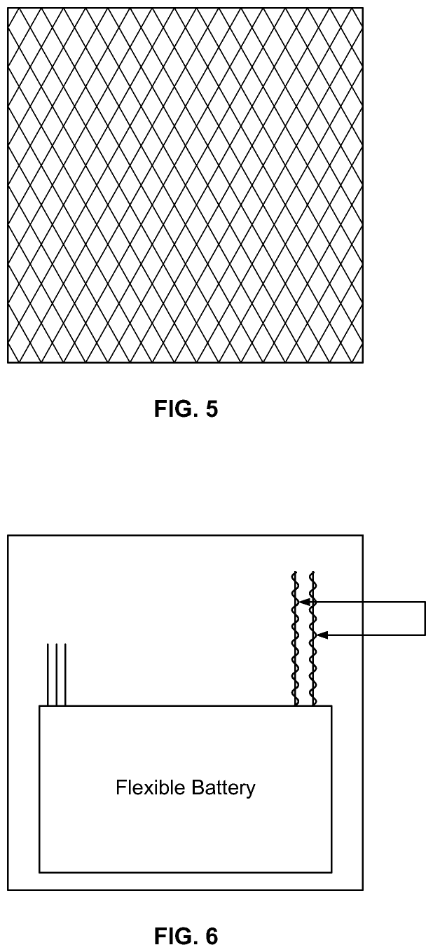

As an example, the monitoring system may include detection units and conductive elastomer panel strips. The panels strips may make up a framework splayed across the garment 12 in diagonal patterns (as is illustrated in FIG. 5, forming a mesh) to provide conductivity to the detection units found on the garment 12. These panel strips may be laminated, woven, and/or stitched to the garment 12 itself. At each contact point/overlap, a detection unit may exist that may create a data packet on the current being passed at that specific monitor. The data packet may include a time at which a current is measured. The detection units may then send the information to either a processor on the garment 12 (e.g., processor 22) or an external processor that may store and analyze the data packets received using either a wired or wireless connection. In some examples, the processor 22 (discussed above) may be included in the monitoring system (and/or may be included on the monitoring layer 50).

Another example of the monitoring system of the monitoring layer 50 is discussed below with regard to FIG. 8.

The power layer 54 (as shown in FIG. 3) may be the innermost layer of the heating and cooling garment system 10. As such, it may be in contact with a user's skin when the heating and cooling garment system 10 is being utilized. In other examples, an additional layer may be added below the power layer 54. The power layer 54 may be made of (or include) a micropolymer embedded plastic.

In the example illustrated in FIG. 6, the power layer 54 includes one or more batteries (e.g., flexible batteries). These batteries may provide power to the monitoring layer 50. Furthermore, these batteries may be utilized to provide electrical current to the Peltier devices 58 in the heating/cooling layer 46. In such an example, the processor 22 may cause electrical current from the batteries to be applied to the Peltier devices 58 in a particular direction. The direction may determine whether the Peltier devices 58 are applying heat or cold to the propagation strips 62 (and thus the heating and cooling zone 38). The amount of current may determine the temperature of the heat or cold. In some examples, the batteries may encompass roughly ⅓ to ½ of the size of the total surface area of the layer 54 (and/or of the garment 12).

FIG. 7 illustrates one example of the insulation layer 46. In the illustrated example, insulation layer 46 includes two materials: an external material and an internal material. The external material (e.g., the material that faces outward) of the insulation layer 46 may be a pliable and/or soft material. Examples of the external material include spandex, microfiber, similar type material(s), or any combination of the preceding. The internal material (e.g., the material that faces inward) of the insulation layer 46 may be a thermal material that may reflect heat back towards the user and/or that may keep the heat in the heating and cooling garment system 10. An example of the internal material includes Biaxially-oriented polyethylene terephthalate (BoPET), such as MYLAR.

FIG. 8 illustrates an alternative example of the monitoring system of the monitoring layer 50. Examples of this monitoring system are included in U.S. Pat. Nos. 9,918,674; 9,793,952; 9,566,033; and U.S. patent application Ser. No. 16/834,821, entitled “Super Modular Monitoring System” and filed on Mar. 30, 2020.

Modifications, additions, or omissions may be made to the heating and cooling garment system 10 without departing from the scope of the disclosure. For example, the heating and cooling garment system 10 may include a monitoring layer 50, or may not include a monitoring layer 50. As another example, in some examples, the batteries and the processor 22 may be included on the heating/cooling layer 46. As a further example, in some examples, the heating and cooling garment system 10 may only be able to apply heat (as opposed to being able to apply either heat or cold), or may only be able to apply cold (as opposed to being able to apply either heat or cold).

This specification has been written with reference to various non-limiting and non-exhaustive embodiments or examples. However, it will be recognized by persons having ordinary skill in the art that various substitutions, modifications, or combinations of any of the disclosed embodiments or examples (or portions thereof) may be made within the scope of this specification. Thus, it is contemplated and understood that this specification supports additional embodiments or examples not expressly set forth in this specification. Such embodiments or examples may be obtained, for example, by combining, modifying, or reorganizing any of the disclosed steps, components, elements, features, aspects, characteristics, limitations, and the like, of the various non-limiting and non-exhaustive embodiments or examples described in this specification.

1. A heating and cooling garment system, comprising:
a garment; and
a plurality of heating and cooling zones positioned within a portion of the garment, each heating and cooling zone being configured to independently heat or cool based on heating and/or cooling instructions received from a mobile application executed on a wireless device.

2. The heating and cooling garment system of claim 1, wherein:
the heating and/or cooling instructions received from the mobile application executed on the wireless device includes an instruction to heat or cool a first heating and cooling zone of the plurality of heating and cooling zones to a first temperature and further includes an instruction to heat or cool a second heating and cooling zone of the plurality of heating and cooling zones to a second different temperature;
the first heating and cooling zone is configured to independently heat or cool to the first temperature based on the heating and/or cooling instructions received from the mobile application executed on the wireless device; and
the second heating and cooling zone is configured to independently heat or cool to the second different temperature based on the heating and/or cooling instructions received from the mobile application executed on the wireless device.

3. The heating and cooling garment system of claim 1, wherein each of the plurality of heating and cooling zones comprises a Peltier heating and cooling device.

4. The heating and cooling garment system of claim 1, wherein each of the plurality of heating and cooling zones comprises:
a Peltier heating and cooling device; and
a propagation strip thermally coupled to a first side of the Peltier heating and cooling device.

5. The heating and cooling garment system of claim 1, wherein the garment is a shirt, pants, a towel, a sleeve, leggings, a mat, or a stretching device.

6. The heating and cooling garment system of claim 1, wherein the plurality of heating and cooling zones are positioned in the portion of the garment.

7. The heating and cooling garment system of claim 1, wherein the plurality of heating and cooling zones are positioned on the portion of the garment.

8. The heating and cooling garment system of claim 1, wherein each heating and cooling zone is configured to independently heat above room temperature based on heating instructions received from the mobile application executed on the wireless device, and wherein each heating and cooling zone is further configured to independently cool below room temperature based on cooling instructions received from the mobile application executed on the wireless device.

9. A method, comprising:
receiving heating and/or cooling instructions from a mobile application executed on a wireless device; and
independently heating or cooling each of a plurality of heating and cooling zones positioned within a portion of a garment, based on the heating and/or cooling instructions.

10. The method of claim 9, wherein the heating and/or cooling instructions received from the mobile application executed on the wireless device includes an instruction to heat or cool a first heating and cooling zone of the plurality of heating and cooling zones to a first temperature and further includes an instruction to heat or cool a second heating and cooling zone of the plurality of heating and cooling zones to a second different temperature; and
wherein independently heating or cooling each of a plurality of heating and cooling zones positioned within the portion of a garment, based on the heating and/or cooling instructions, comprises:
independently heating or cooling the first heating and cooling zone to the first temperature based on the heating and/or cooling instructions received from the mobile application executed on the wireless device; and
independently heating or cooling the second heating and cooling zone to the second different temperature based on the heating and/or cooling instructions received from the mobile application executed on the wireless device.

11. The method of claim 9, wherein each of the plurality of heating and cooling zones comprises:
a Peltier heating and cooling device; and
a propagation strip thermally coupled to a first side of the Peltier heating and cooling device.

12. The method of claim 9, wherein the garment is a shirt, pants, a towel, a sleeve, leggings, a mat, or a stretching device.

13. The method of claim 9, wherein the heating and/or cooling instructions received from the mobile application executed on the wireless device comprise heating instructions;
wherein independently heating or cooling each of a plurality of heating and cooling zones positioned within the portion of the garment, based on the heating and/or cooling instructions, comprises independently heating above room temperature each of the plurality of heating and cooling zones positioned within the portion of the garment, based on the heating instructions; and
wherein the method further comprises:
receiving cooling instructions from the mobile application executed on the wireless device; and
independently cooling below room temperature each of the plurality of heating and cooling zones positioned within the portion of the garment, based on the cooling instructions.

14. A heating and cooling sleeve, comprising:
a sleeve configured to be in contact with a user's skin;
a plurality of heating and cooling zones positioned in series within the sleeve, each heating and cooling zone comprising:
a Peltier heating and cooling device; and
a propagation strip thermally coupled to a first side of the Peltier heating and cooling device;
one or more processors positioned within the sleeve and operable, upon execution, to receive heating and/or cooling instructions from a wireless device, wherein the heating and/or cooling instructions include an instruction to heat or cool a first heating and cooling zone of the plurality of heating and cooling zones to a first temperature and further include an instruction to heat or cool a second heating and cooling zone of the plurality of heating and cooling zones to a second different temperature;
wherein the first heating and cooling zone is configured to independently heat or cool to the first temperature based on the heating and/or cooling instructions received from the wireless device; and
wherein the second heating and cooling zone is configured to independently heat or cool to the second different temperature based on the heating and/or cooling instructions received from the wireless device.

15. The heating and cooling sleeve of claim 14, wherein the plurality of heating and cooling zones are positioned in series in the sleeve; and wherein the one or more processors are positioned in the sleeve.

16. The heating and cooling sleeve of claim 14, wherein the sleeve comprises a plurality of layers, wherein the plurality of heating and cooling zones are positioned on a first layer of the plurality of layers, and wherein the one or more processors are positioned on a second layer of the plurality of layers.

17. The heating and cooling sleeve of claim 14, wherein the heating and/or cooling instructions include an instruction to heat or cool a third heating and cooling zone of the plurality of heating and cooling zones to a third different temperature that is different than the first temperature and the second different temperature, wherein the third heating and cooling zone is configured to independently heat or cool to the third different temperature based on the heating and/or cooling instructions received from the wireless device.

18. The heating and cooling sleeve of claim 14, wherein the one or more processors are operable, upon execution, to receive the heating and/or cooling instructions from a mobile application executed on the wireless device.

19. The heating and cooling sleeve of claim 14, further comprising one or more batteries positioned within the sleeve and configured to provide electrical current to each of the Peltier heating and cooling devices.

20. The heating and cooling sleeve of claim 14, wherein:
the heating and/or cooling instructions include an instruction to heat the first heating and cooling zone to a first temperature above room temperature;
the first heating and cooling zone is configured to independently heat to the first temperature above room temperature based on the heating and/or cooling instructions received from the wireless device;
the one or more processors are further operable, upon execution, to receive second heating and/or cooling instructions from the wireless device, wherein the second heating and/or cooling instructions include an instruction to cool the first heating and cooling zone to a third temperature below room temperature; and
the first heating and cooling zone is further configured to independently cool to the third temperature below room temperature based on the second heating and/or cooling instructions received from the wireless device.
